(19) United States
(12) Patent Application Publication
(10) Pub. No.: US 2018/0206526 A1
(43) Pub. Date: Jul. 26, 2018

(54) APPLYING MEASUREMENT, CONTROL AND AUTOMATION TO A DRY CORN MILLING ETHANOL PRODUCTION PROCESS TO MAXIMIZE THE RECOVERY OF ETHANOL AND CO-PRODUCTS

(71) Applicant: CIDRA CORPORATE SERVICES INC., Wallingford, CT (US)

(72) Inventors: James T. TURNBULL, Madison, MS (US); Bret H. MAGNESS, Collierville, TN (US)

(21) Appl. No.: 15/745,818

(22) PCT Filed: Jul. 20, 2016
(86) PCT No.: PCT/US2016/043122

§ 371 Date: Jan. 18, 2018

Related U.S. Application Data
(60) Provisional application No. 62/194,539, filed on Jul. 20, 2015.

(51) Int. Cl. A23K 10/38; C12P 7/06; C11B 13/00; C12M 1/00

(52) U.S. Cl. CPC A23K 10/38; C12P 7/06; C12M 45/02; C12M 21/12; C12M 43/02; C11B 13/00

(57) ABSTRACT

Apparatus features a signal processor or signal processing module configured to: receive signaling containing information about about a measurement of one or more constituents of an output stream from a centrifuge in a dry corn milling process, including to produce Ethanol; and determine corresponding signaling containing information about a real time feedback control of the dry corn milling process, based upon the signaling received. The signal processor or signal processing module is configured to provide the corresponding signaling as control signaling to provide the real time feedback control of the dry corn milling process.

### CROSS-REFERENCE TO RELATED APPLICATIONS

This application claims benefit to provisional patent application Ser. No. 62/194,539 (712-2.423//CCS-0147), filed 20 Jul. 2015; which is incorporated by reference in its entirety.

### BACKGROUND OF THE INVENTION

### 1. Field of Invention

This invention relates to a technique for controlling a dry corn milling process; and more particularly relates to a technique for controlling a dry corn milling ethanol production process.

### 2. Description of Related Art

A dry corn milling process is the predominant method of Ethanol production in North America. In operation, dry corn mills generate ethanol, corn syrup and corn oil from corn via fermentation, distillation and separation processes. After the distillation phase that follows the corn fermentation process, two marketable/valuable by-products, corn oil and corn syrup, are produced via evaporation and separation of the stillage by-products. Stillage processing is augmented by emulsion breaking chemistries that improve the rate and efficiency at which the corn oil and corn syrup phases are separated. The separation process performance is monitored periodically off-line by gravimetrically measuring the purity of the corn oil and syrup streams. Using the dry corn milling process to produce Ethanol, the production of co-products can make the difference between a profitable and unprofitable production operation.

A number of measurements are utilized in a dry corn milling process to test the efficacy of co-product production. For example, oil content in the discharges of a cyclone separator may be tested once or twice a day. However, the results of the test may take hours, and this does not provide a real time feedback for controlling the process. Additionally, there are a number of additional measurements that, if applied to the dry milling process, could help control and optimize the process. The known measurement processes in the art are carried out manually, and are not carried out as part of an automated process.

In view of this, there is a need in the industry for a better way for controlling a dry corn milling process, including to produce Ethanol.

### SUMMARY OF THE INVENTION

In summary, the present invention provides a combination of in-line “real-time” measurements in a dry corn milling process, so that immediate feedback can be provided in the dry corn milling process to optimize the production of Ethanol and co-products, including CO² and corn oil. The present invention provides techniques of enhancing the efficiency of ethanol fermentation, distillation and valuable by-product production (corn oil and corn syrup), e.g., that feature automating the ethanol fermentation, distillation and separation of corn oil and corn syrup from the stillage.

By way of example, the new and unique techniques, e.g., may include, or take the form of, a method and/or an apparatus, to provide a real time feedback control of a dry corn milling process, including to produce Ethanol.

According to some embodiments of the present invention, the apparatus may may feature at least one signal processor or signal processing module configured at least to:
receive signaling containing information about a measurement of one or more constituents of an output stream from a centrifuge in a dry corn milling process, including to produce Ethanol; and
determine corresponding signaling containing information about a real time feedback control of the dry corn milling process, based upon the signaling received.

The apparatus may include one or more of the following additional features:

The signal processor or processing module may be configured to provide the corresponding signaling, e.g., as control signaling to provide the real time feedback control of the dry corn milling process.

The one or more constituents of the output stream may be selected from the following group: corn oil, corn syrup, one or more proteins, and leftover yeast solids.

The measurement of the one or more constituents of the output stream may be selected from the following group: the purity of corn oil in the output stream, including the fat content; the purity of the syrup in the output stream, including the carbohydrate content; the amount of protein in the output stream, including proteins useful in animal foods; the amount of water in the output stream; and the amount of leftover yeast solids in the output stream.

The measurement of the one or more constituents of the output stream may be based upon an optical interrogation of the output stream.

The optical interrogation of the output stream may include processing optical signaling provided and/or sensed in relation to the output stream.

The optical interrogation of the output stream may include using a near-infrared spectroscopy technique for optically interrogating the output stream.

The optical interrogation of the output stream may include using a Raman optical scattering technique for optically interrogating the output stream.

The apparatus may include an optical measurement/interrogation device configured to provide optical interrogation signaling, receive sensed optical interrogation signaling containing information about the one or more constituents of the output stream and provide the signaling, e.g., to the signal processor or processing module. The optical measurement/interrogation device may include an optical probe or other suitable optical sensing device.

The measurement of the one or more constituents of the output stream may be based upon a chemical interrogation of the output stream.

The chemical interrogation of the output stream may include processing a sample/portion of the output stream based upon determining a chemical content of the sample/portion of the output stream.

The apparatus may include a chemical measurement device configured to receive the sample/portion of the output stream containing the one or more constituents of the output stream, process the sample/portion and provide the signaling containing chemical interrogation information about the one or more constituents of the output stream, e.g., to the signal processor or processing module.

The real time feedback control of the dry corn milling process may include providing a control signal to adjust one or more parameters of the dry corn milling process, including where the control signal is used to control the centrifuge, a backend process such as a fermentation process, or both.

The one or more parameters may include some combination of the following: a chemical parameter adjustment to one or more sub-processes in the dry corn milling process, including an adjustment to the amount, or timing, or location, of a de-emulsifier dosed to the centrifuge; or a lever parameter adjustment of one or more components in the dry corn milling process, including adjusting a control or throughput lever in the centrifuge; or a throughput parameter adjustment of one or more components in the dry corn milling process, including adjusting a throughput in the centrifuge; or a flow parameter adjustment in the one or more components in the dry corn milling process, including adjusting a flow parameter of the centrifuge; or a cycle time parameter adjustment in the one or more components in the dry corn milling process, including varying the cycle time of the centrifuge; or a backflush parameter adjustment in the one or more components in the dry corn milling process, including setting up backflush cycles for the centrifuge; or a diversion parameter adjustment in the one or more components in the dry corn milling process, including diverting a portion of the output stream of the centrifuge to another component in the dry corn milling process.

The centrifuge may be configured to receive an input stream containing syrup/oil and provide a co-product stream containing the one or more constituents having corn oil and light, low density solids.

The centrifuge may be configured to receive the input stream and provide a second co-product stream containing the one or more constituents having corn syrup and higher density solids.

The Raman optical scattering technique may include comparing a sensed optical scattering signaling in the output stream to a signature optical scattering signaling stored in an optical scattering database and determining the purity of corn oil in the output stream based upon the comparison.

The real time feedback control of the dry corn milling process may include sensing a desired level of corn oil capture, and providing the control signal to divert a portion of corn oil from the output stream to a distillation process in the dry corn milling process for producing dried distillers grain (DDGs) that goes into animal feed to increase its fat content.

The real time feedback control of the dry corn milling process may include controlling a split of corn oil and corn syrup provided from the centrifuge, and determining whether to feed either the corn oil or the corn syrup back to the centrifuge for further purification, or claim the corn oil, or sending some portion of the corn oil to a backend process of the dry corn milling process.

According to some other embodiments, the present invention may take the form of a method featuring steps for receiving in a signal processor or signal processing module signaling containing information about a measurement of one or more constituents of an output stream from a centrifuge in a dry corn milling process, including to produce Ethanol; and determining in the signal processor or signal processing module corresponding signaling containing information about a real time feedback control of the dry corn milling process, based upon the signaling received.

The signal processor or signal processor module may take the form of a signal processor and at least one memory including a computer program code, where the signal processor and at least one memory are configured to cause the apparatus to implement the functionality of the present invention, e.g., to respond to signaling received and to determine the corresponding signaling, based upon the signaling received.

According to some embodiment, the present invention may take the form of apparatus comprising means for receiving signaling containing information about a measurement of one or more constituents of an output stream from a centrifuge in a dry corn milling process, including to produce Ethanol; and means for determining corresponding signaling containing information about a real time feedback control of the dry corn milling process, based upon the signaling received, consistent with that set forth herein.

According to some embodiments of the present invention, the apparatus may also take the form of a computer-readable storage medium having computer-executable components for performing the steps of the aforementioned method. The computer-readable storage medium may also include one or more of the features set forth above.

In effect, the present invention is directed at increasing the efficiency of fermentation, distillation and separation via real time automation. In operation, the present invention generates real time, on-line separation effectiveness data that provides control targets for prior processing stage variables. These include, but are not limited to, yeast application, fermentation temperature and dwell time, centrifuge speed and maintenance protocol, de-emulsifier application strategy and dosage, and classification of final product quality. Overall, the present invention provides a better way for controlling a dry corn milling process, including to produce Ethanol.

### BRIEF DESCRIPTION OF THE DRAWING

The drawing includes FIGS. 1-7, which are not necessarily drawn to scale, as follows:

FIG. 1 shows a block diagram of apparatus, e.g., having a signal processor or signal processing module for implementing signal processing functionality, according to some embodiments of the present invention.

FIG. 2 is a block diagram of part of a corn milling process, e.g., to produce Ethanol, that may form part of some embodiments of the present invention.

FIG. 3 is a block diagram of another part of a corn milling process, e.g., that may form part of some embodiments of the present invention.

FIG. 4 is a diagram of a centrifuge that may form part of the separation step shown in FIG. 3, e.g., according to some embodiments of the present invention.

FIG. 5A is a separator/centrifuge inlet data process flow diagram, e.g., according to some embodiments of the present invention.

FIG. 5B is a separator/centrifuge outlet data process flow diagram, e.g., according to some embodiments of the present invention.

FIG. 6 is an evaporator inlet data process flow diagram, e.g., according to some embodiments of the present invention.

FIG. 7 is a beer well data process flow diagram, e.g., according to some embodiments of the present invention.

### DETAILED DESCRIPTION OF BEST MODE OF THE INVENTION

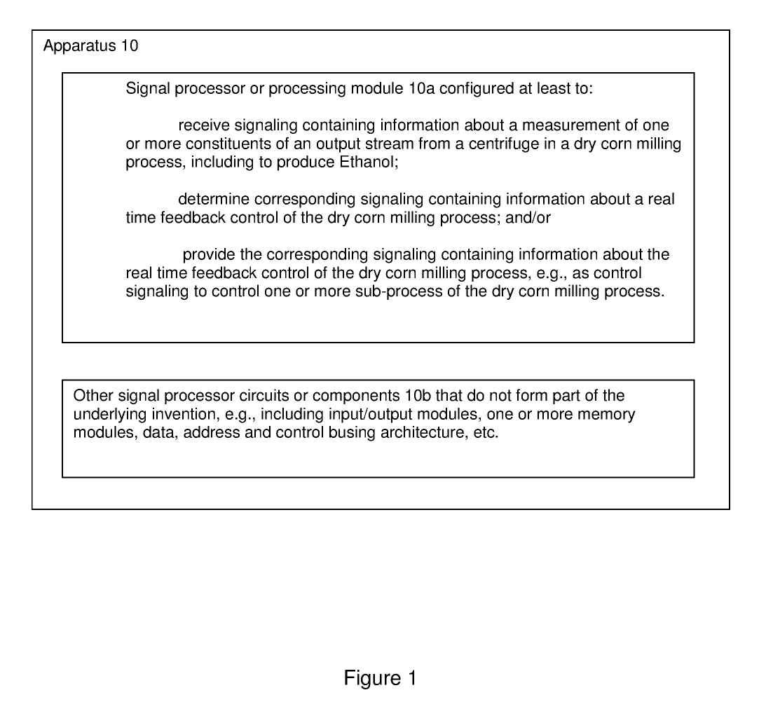

### FIG. 1: Basic Invention

By way of example, FIG. 1 shows apparatus 10, e.g. having at least one signal processor or signal processing module 10a for implementing the signal processing functionality according to the present invention. In operation, the at least one signal processor or signal processing module 10a may be configured at least to:
receive signaling containing information about a measurement of one or more constituents of an output stream from a centrifuge in a dry corn milling process, including to produce Ethanol; and
determine corresponding signaling containing information about a real time feedback control of the dry corn milling process, based upon the signaling received.

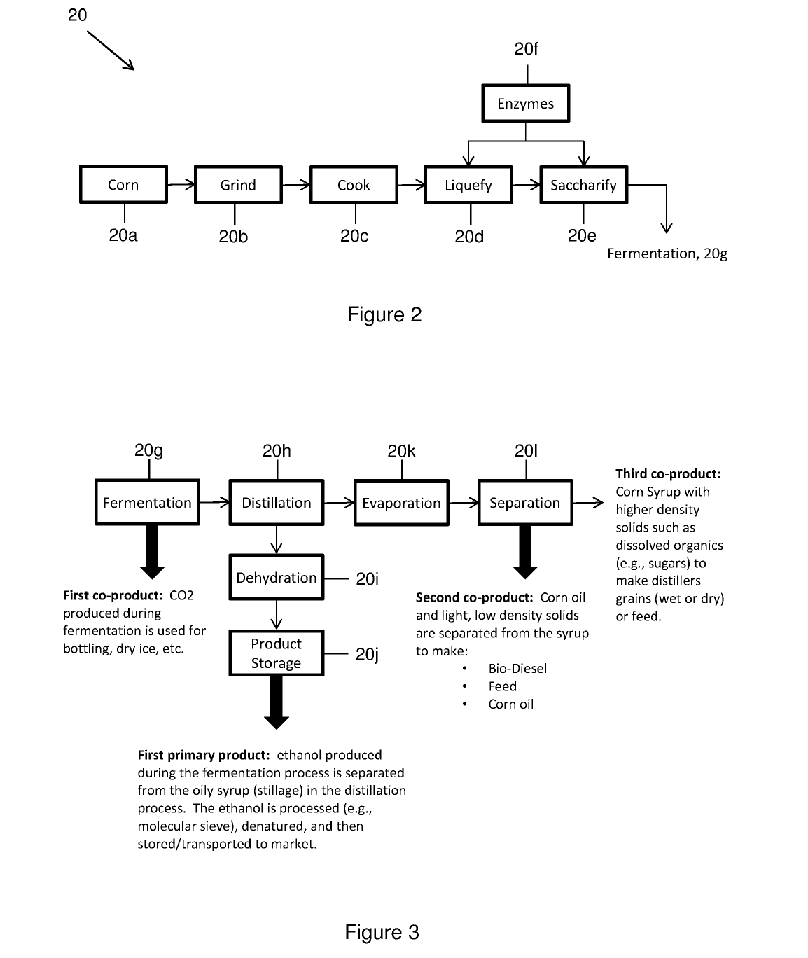

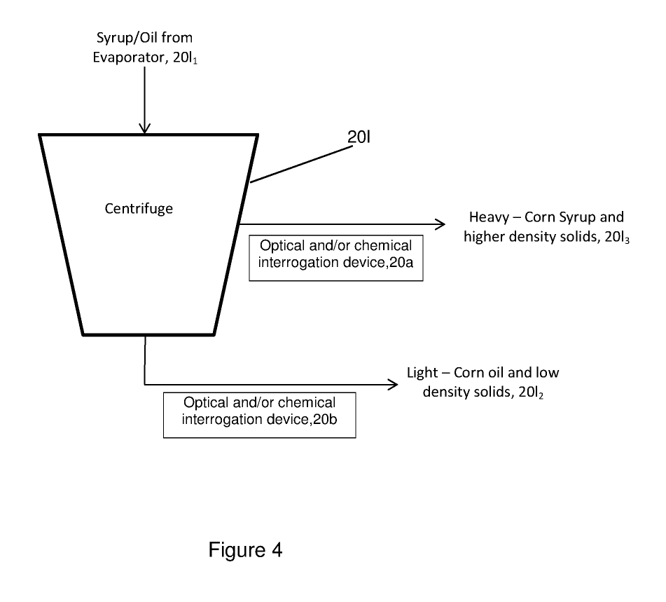

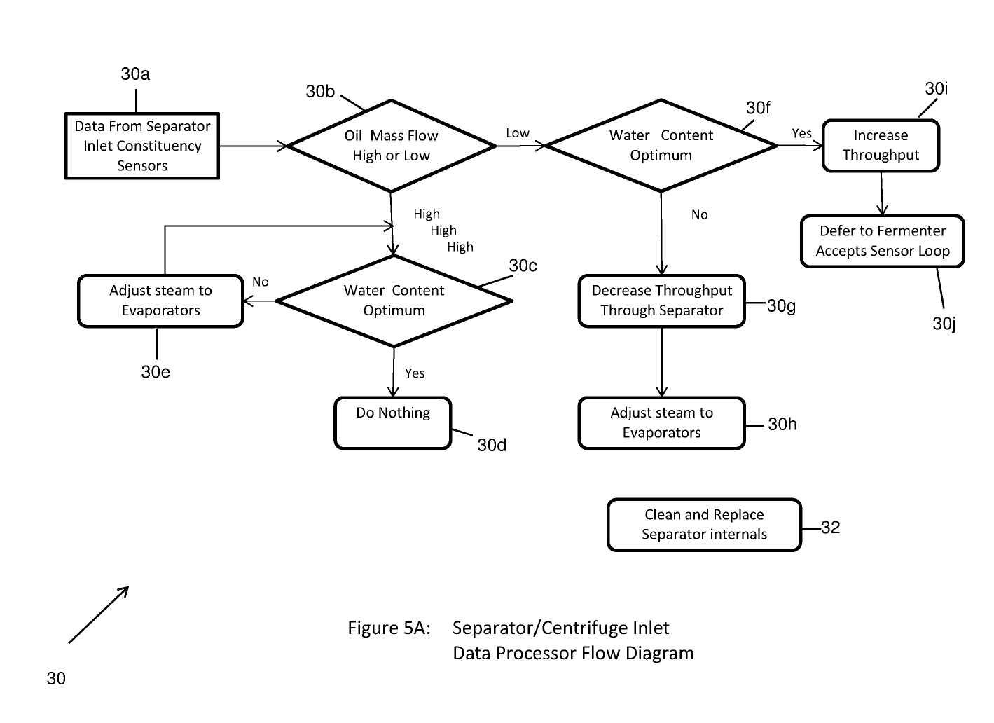

By way of example, the output stream may include either output stream 20l₂ or 20l₃ of a centrifuge 20l (FIG. 4) that forms part of a dry corn milling process shown in FIGS. 2-3, consistent with that set forth below.

The at least one signal processor or signal processing module 10a may be configured to provide the corresponding signaling, e.g., as control signaling to provide or implement the real time feedback control of the dry corn milling process, e.g., consistent with that set forth herein. By way of example, the control signaling may provide the real time feedback control of the dry corn milling process that form part of that shown in FIGS. 2-7, consistent with that set forth herein.

The functionality of the signal processor or processor module 10a may be implemented using hardware, software, firmware, or a combination thereof. In a typical software implementation, the processor module may include one or more microprocessor-based architectures having a microprocessor, a random access memory (RAM), a read only memory (ROM), input/output devices and control, data and address buses connecting the same, e.g., consistent with that shown in FIG. 1, e.g., see element 10b. A person skilled in the art would be able to program such a microprocessor-based architecture(s) to perform and implement such signal processing functionality described herein without undue experimentation. The scope of the invention is not intended to be limited to any particular implementation using any such microprocessor-based architecture or technology either now known or later developed in the future, or any particular way of programming the signal processor to implement the signal processing functionality according to the present invention.

By way of example, the apparatus 10 may also include, e.g., other signal processor circuits or components 10b that do not form part of the underlying invention, e.g., including input/output modules, one or more memory modules, data, address and control busing architecture, etc. In operation, the at least one signal processor or signal processing module 10a may cooperation and exchange suitable data, address and control signaling with the other signal processor circuits or components 10b in order to implement the signal processing functionality according to the present invention. By way of example, the signaling may be received by such an input module, provided along such a data bus and stored in such a memory module for later processing, e.g., by the at least one signal processor or signal processing module 10a. After such later processing, processed signaling resulting from any such determination may be stored in such a memory module, provided from such a memory module along such a data bus to such an output module, then provided from such an output module as the corresponding signaling, e.g., by the at least one signal processor or signal processing module 10a.

The scope of the invention is not intended to be limited to any particular implementation using technology either now known or later developed in the future.

The scope of the invention is intended to include implementing the functionality of the processors 10a as stand-alone processor, signal processor, or signal processor module, as well as separate processor or processor modules, as well as some combination thereof. By way of example, a person skilled in the art would appreciate and understanding without undue experimentation, especially after reading the instant patent application together with that known in the art, e.g., how to implement suitable signaling processing functionality to receive the signaling containing information about the measurement of the one or more constituents of the output stream 20l₂ or 20l₃ of the centrifuge 20l (FIG. 4), e.g., using signal processing techniques that are either now known or later developed in the future. Consistent with that described herein, the signaling may include, or take the form of, suitable signaling containing information about an optical or chemical interrogation of the output stream 20l₂ or 20l₃ of the centrifuge 20l (FIG. 4) that contains information about the measurement of the one or more constituents of the output stream 20l₂ or 20l₃.

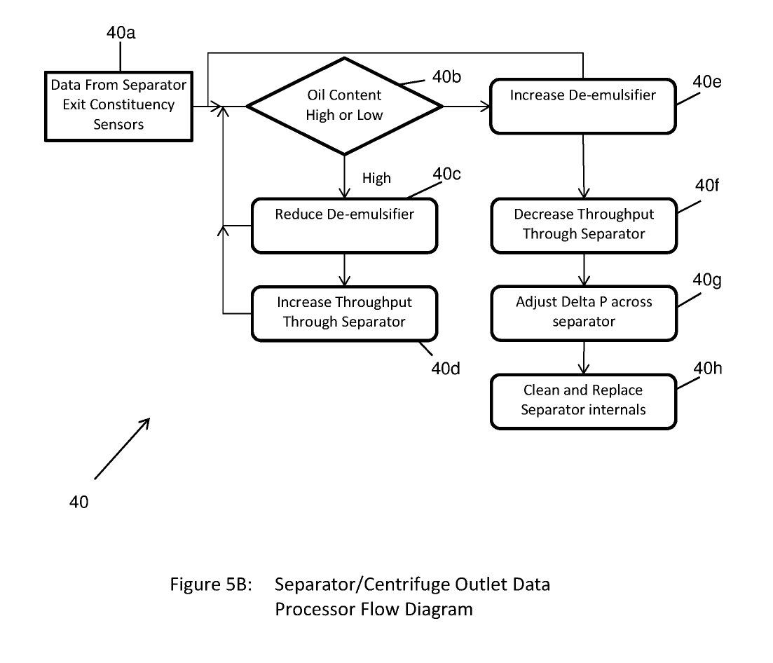

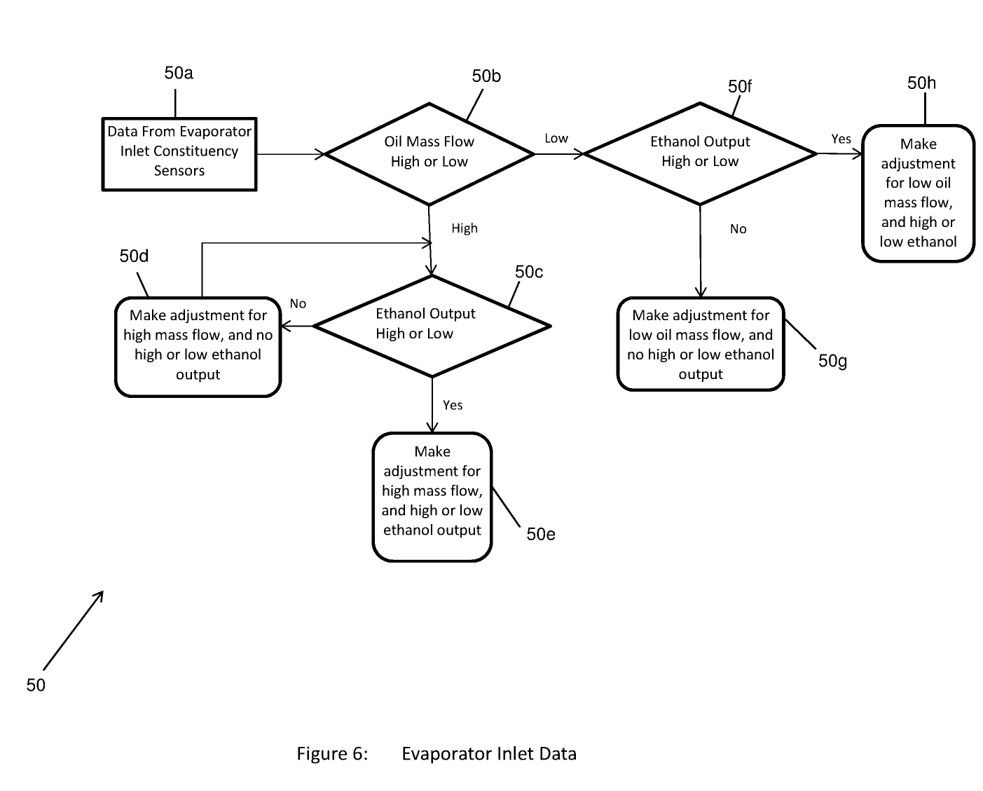

By way of further example, a person skilled in the art would appreciate and understanding without undue experimentation, especially after reading the instant patent application together with that known in the art, e.g., how to implement suitable signaling processing functionality to make one or more such determinations, based upon the signaling received, using signal processing techniques that are either now known or later developed in the future. Consistent with that described herein, the signal processing determination may include, or take the form of, implementing one or more of the steps show in FIGS. 5A, 5B, 6 and/or 7.

### FIGS. 2-7: Examples of Measurement, Control and Automation in the Dry Corn Milling Process

By way of example, and consistent with that shown in FIGS. 2-7, the present invention may be implemented by applying measurement, control and automation to a dry corn milling ethanol production process to maximize the recovery of ethanol and co-products. The dry corn milling process ethanol production process is disclosed herein, and may include a dry corn milling process and a measurement, control and automation of the separation process, e.g., consistent with that as follows:

### Summary of the Dry Corn Milling Process

FIGS. 2 and 3 show steps that form part of the dry corn milling process generally indicated as 20 (FIGS. 2) and 30 (FIG. 3).

1. Grain Storage 20a—In step 20a, the corn is stored for milling.

2. Corn Milling 20b—In step 20b, the corn is ground into powder (“Meal”).

3. Cooking and Liquefaction 20c, 20d, 20e and 20f—In steps 20c, 20d, 20e and 20f, the dry milled corn is prepared for fermentation. For example, the Meal is mixed with water to form a “Mash”, where the starch is converted into dextrose. Ammonia may be added for pH control and as a nutrient for yeast. The Mash is cooked to reduce bacteria, the cooled and transferred to a fermenter.

4. Fermentation to create Ethanol (added yeast): In step 20g, yeast is added and the sugars are converted into ethanol and CO₂, e.g., so as to form a first co-product.

a. The first co-product: CO₂ produced during fermentation is used for bottling, dry ice, etc.

b. The remainder of the fermented corn mill is further processed in steps 20h through 20l.

5. Distillation 20h: In step 20h, Ethanol is separated so as to form a first primary product, and what remains is stillage.

a. The First primary product: In steps 20h, ethanol produced during the fermentation process is separated from the oily syrup (stillage) in the distillation process. In steps, 20i and 20j, the ethanol is processed (e.g., using a molecular sieve), denatured, and then stored/transported to the market.

b. The remainder of the distilled corn mill is stillage (syrup/oil mix) that is further processed in steps 20k through 20l.

6. The stillage (syrup/oil mix) is processed in step 20k through evaporation, tank storage, strainer to a separator/centrifuge in step 20l to separate the components. After evaporation, the stillage is in a range of about 3% to 5% oil.

7. A process aid (de-emulsifier) may be added to the evaporated stillage prior to being provided to the separator/centrifuge process 20l to aid in the water/oil separation. The separator/centrifuge receives the syrup/oil 20l₁ from the evaporator, which is processed via the separation/centrifuge process 20l to provide a first output stream 20l₂ in the form of a Second co-product and a second output stream 20l₃ in the form of a third co-product.

a. The Second co-product: Corn oil and light, low density solids are separated from the syrup and provided as the first output stream 20l₂, which may be used to make:
i. Bio-Diesel.
ii. Feed.
iii. Corn oil.

b. The Third co-product: Corn Syrup with higher density solids, such as dissolved organics (e.g., sugars), are separated and provided as the second output stream 20l₃, which may be used to make:
i. Distillers grains (wet or dry) for feed.

### Input/Output Stream Interrogation Techniques

By way of example, one or more of the output streams 20l₂ and/or 20l₃ may be interrogated in order to determine the information about the one or more constituents of the one or more output streams therein. The interrogation may include, or take the form of, optical and/or chemical interrogation, e.g., by using an optical and/or chemical interrogation device 21a and/or 21b like that shown in FIG. 4, e.g., configured in relation to piping/conduit that receives the one or more of the output streams 20l₂ and/or 20l₃. (The lines/arrows in FIG. 4 are understood to represent piping/conduit having the one or more of the output streams 20l₂ and/or 20l₃ flowing therein.)

By way of further example, an optical interrogation device like element 21a, 21b may include, or take the form of, an optical probe to provide optical interrogation signaling and an optical sensor to receive sensed optical interrogation signaling passing through, or reflected by, an output stream like streams 20l₂ and/or 20l₃, and provide optical interrogation signaling containing the optical interrogation information about the one or more constituents of the output stream. The optical interrogation technique may include, or take the form of, using a near-infrared spectroscopy technique, a Raman scattering technique, as well as other types or kinds of optical interrogation techniques that are either now known or later developed in the future.

Alternatively, and by way of further example, a chemical interrogation device like element 21a, 21b may include, or take the form of, a chemical probe to receive a chemical interrogation sample and a chemical sensor to process the chemical interrogation sample and provide chemical interrogation signaling containing the chemical interrogation information about the one or more constituents of the output stream. The chemical interrogation technique may include, or take the form of, using known chemical interrogation techniques to determine the present of the one or more constituents set forth herein, as well as other types or kinds of chemical interrogation techniques that may be later developed in the future to determine the present of the one or more constituents set forth herein.

Alternatively, and by way of still further example, the scope of the invention is intended to include using other types or kind of interrogation techniques to determine the present of the one or more constituents set forth herein, that are either now known or later developed in the future.

By way of example, a similar interrogation device like element 21a, 21b may be configured or implemented in relation to the input stream 20l₁ flowing into the separator 20l, e.g., consistent with that set forth herein.

### FIG. 5A and 5B: Measurement, Control and Automation of Separation Process

FIG. 5A shows a separator/centrifuge inlet data process flow diagram having a flowchart generally indicated as 30 with steps 30a through 30j, e.g., according to some embodiments of the present invention. The steps may include the following:

a step 30a for receiving data from a separator inlet constituency sensor of the separator 20l (FIG. 4), e.g., similar to the elements 20a, 20b configured in relation to the input flow stream 20l₁ of syrup/oil from the evaporator 20k into the separator 20l;

a step 30b for determining if the oil mass flow is high or low;
if the oil mass flow is high, then
   a step 30c for determining if the water content is optimum;
   a step 30d for doing nothing (i.e., no process adjustment) if the water content is optimum;
   a step 30e for adjusting the steam to the evaporator(s) 20k if the the water content is not optimum (and repeating step 30c when needed); alternatively, if the oil mass flow is low, then
   a step 30f for determining if the water content is optimum;
   a step 30g for decreasing the throughput through the separator 20l if the water content is not optimum, e.g., by sending control signaling for adjusting a centrifuge flow level or valve;
   a step 30h for adjusting the steam to the evaporator(s) 20k if the water content is not optimum, e.g., by sending control signaling for adjusting an evaporator flow level or valve;
   a step 30i for increasing the throughput through the separator 20l if the water content is optimum, e.g., by sending control signaling for adjusting a centrifuge flow level or valve; and
   a step 30j for deferring to the fermenter's accepts sensor loop if the water content is optimum.

The flowchart 30 may also include a step 32 for cleaning and replacing separator internals, e.g., based upon the signaling sensed, as well as part of a routine maintenance procedure.

FIG. 5B is a separator/centrifuge outlet data process flow diagram, having a flowchart generally indicated as 40 with steps 40a through 40h, e.g., according to some embodiments of the present invention. The steps may include the following:

a step 40a for receiving data from a separator exit constituency sensor of the separator 20l (FIG. 4), e.g., that may include the elements 20a, 20b configured in relation to the output stream 20l₂ of light corn oil and low density solids, or the output stream 20l₃ of heavy corn syrup and higher density solids, provided from the separator 20l;

a step 40b for determining if the oil content is high or low;
if the oil content is high, then
   a step 40c for reducing the de-emulsifier, e.g., by sending control signaling for adjusting a de-emulsifier flow valve;
   a step 40d for increasing the throughout through the separator 20l, e.g., by sending control signaling for adjusting a centrifuge flow level or valve;
alternatively, if the oil content is low, then
   a step 40e for increasing the de-emulsifier provided to the separator 20l (and repeating step 40b when needed);
   a step 40f for decreasing the throughput through the separator 20l;
   a step 40g for adjusting the Delta P across the separator 20l;
   a step 40h for cleaning and replacing separator internals.

One or more of the steps in FIGS. 5A and 5B may be implemented in whole or in part by the signal processor or processing module 10a and circuits/components 10b shown in FIG. 1, e.g., including providing the control signaling.

### FIG. 6: Measurement, Control and Automation of the Evaporator Process

FIG. 6 shows an evaporator inlet data process flow diagram having a flowchart generally indicated as 50 with steps 50a through 50h, e.g., according to some embodiments of the present invention. The steps may include the following:

a step 50a for receiving data from an evaporator inlet constituency sensor(s) of the evaporator 20k (FIG. 3), e.g., similar to the elements 20a, 20b but configured in relation to the input flow of the evaporator 20k;

a step 50b for determining if the oil mass flow is high or low;
if the oil mass flow is high, then
   a step 50c for determining if the ethanol output is high or low;
   a step 50d for making an adjustment in the case for high mass flow, and no high or low ethanol output (and repeating step 50c when needed);
   a step 50e for making an adjustment in the case for high mass flow, and high or low ethanol output;
alternatively, if the oil mass flow is low, then
   a step 50f for determining if the ethanol output is high or low;
   a step 50g for making an adjustment in the case for low oil mass flow, and no high or low ethanol output; and
   a step 50h for making an adjustment in the case for low oil mass flow, and high or low ethanol output.

One or more of the steps in FIG. 6 may be implemented in whole or in part by the signal processor or processing module 10a and circuits/components 10b shown in FIG. 1, including providing the control signaling.

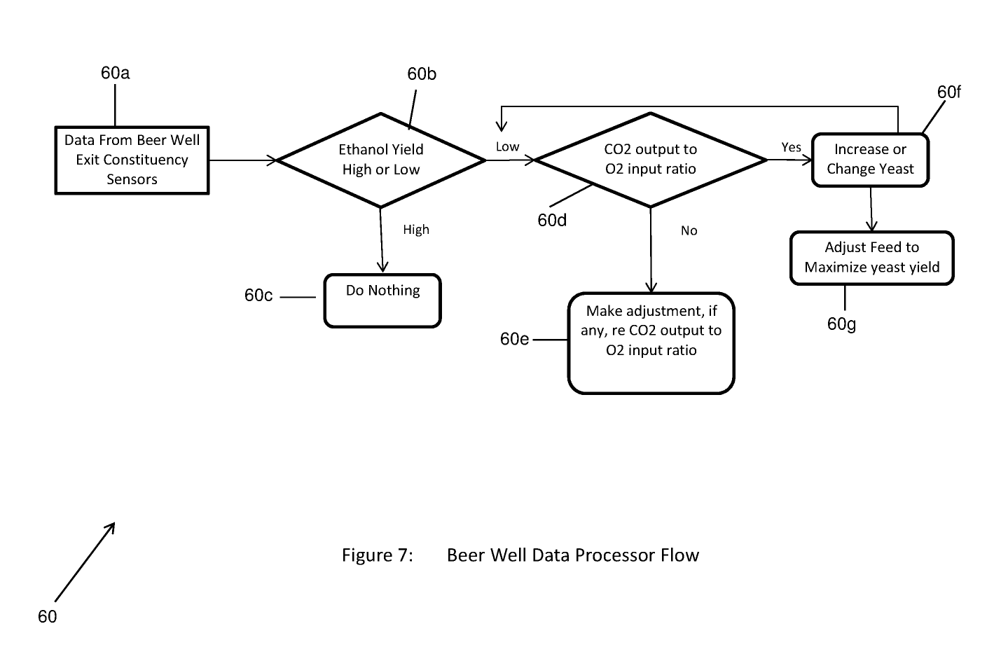

### FIG. 7: Measurement, Control and Automation of the Beer Well Process

FIG. 7 shows a beer well data process flow diagram having a flowchart generally indicated as 60 with steps 60a through 60g, e.g., according to some embodiments of the present invention. The steps may include the following:

a step 60a for receiving data from a beer well exit constituency sensor(s), not shown;

a step 60b for determining if the ethanol yield is high or low;
if the ethanol yield is high, then implementing a step 60c for doing nothing;
alternatively, if the ethanol yield is low, then
   a step 60d for determining a CO₂ output to O₂ input ratio;
   a step 60e for making an adjustment, if any, including no adjustment, in relation to the ratio determined; and
   a step 60f for increasing or changing the yeast in relation to the ratio determined.

One or more of the steps in FIG. 7 may be implemented in whole or in part by the signal processor or processing module 10a and circuits/components 10b shown in FIG. 1.

### Examples of Measurement, Control and Automation of Process

By way of example, and consistent with that set forth in FIGS. 5A, 5B, 6 and 7, the measurement, control and automation of the overall process may include implementing one or more of the following:

1. Measurement of Oil Content:

By way of example, the measurement of oil content may include:
a. Feedstock—In the stillage after evaporation-input to the cyclone.
b. Lite oil and low density solids output of the cyclone 20l (FIG. 4).
c. De-oiled heavy syrup and high density solids output of the cyclone.

2. Measurement of Water Content:

By way of example, the measurement of water content may include:
a. Feedstock—In the stillage after evaporation-input to the cyclone.
b. Lite oil and low density solids output of the cyclone.
c. De-oiled heavy syrup and high density solids output of the cyclone.

3. Measure of Solids Content:

The measure of solids content may include:
a. Feedstock—In the stillage after evaporation-input to the cyclone.
b. Lite oil and low density solids output of the cyclone.
c. De-oiled heavy syrup and high density solids output of the cyclone.

4. Measure of Sugar Content:

By way of example, the measure of sugar content may include:
a. At the feed to the fermenter.
b. At the discharge of the fermenter.

5. Measure Alcohol Content:

By way of example, the measure of alcohol content may include:
a. Between fermentation stages.
b. At the discharge of the fermenter.

6. Measurement of Air (GVF) Content:

By way of example, the measurement of air (GVF) content may include:
a. Feedstock—In the stillage after evaporation-input to the cyclone.
b. Lite oil and low density solids output of the cyclone.
c. De-oiled heavy syrup and high density solids output of the cyclone.
d. Measurement of the fermentation process-control gas content to prevent venting and to maximize the recovery of CO₂.

7. Control Based on Measurement:

By way of example, the control based on measurement(s) may include one or more of the following adjustments:
a. Adjust the set up and/or cleaning schedule of the separator based on observed performance.
b. Adjust the speed of the dosing pump feeding de-emulsifier chemistry to the process—As oil purity goes up, de-emulsifier dose would be decreased and vice-versa.
c. Adjust feed rate of process liquid to the separator.
d. Adjust the set up and/or cleaning schedule of the fermenter.
e. Adjust the dosing of defoamer and/or deaeration chemistry to the fermenter to control CO₂ production.
f. Adjust the dosing of yeast to optimize CO₂ production and/or reduce measured sugars output to the distillation.
g. Adjusting yeast, enzyme addition and air addition to the fermentation stages based on the measurement of alcohol content between stages and at the discharge of the last fermenter.
h. Adjust dosing of air in the yeast activation phase (propagators) prior to introduction to the fermentation stage.

Consistent with that set forth herein, one or more of the measurements may be used to control and automate the separation process, as well as any of the other processes or sub-processes used to process the milled dry corn, including the fermentation process/stage.

### The Scope of the Invention

While the invention has been described with reference to an exemplary embodiment, it will be understood by those skilled in the art that various changes may be made and equivalents may be substituted for elements thereof without departing from the scope of the invention. In addition, may modifications may be made to adapt a particular situation or material to the teachings of the invention without departing from the essential scope thereof. Therefore, it is intended that the invention not be limited to the particular embodiment(s) disclosed herein as the best mode contemplated for carrying out this invention.

What is claimed is:

1. Apparatus comprising:
a signal processor or processing module configured at least to:
receive signaling containing information about a measurement of one or more constituents of an output stream from a centrifuge in a dry corn milling process, including to produce Ethanol; and
determine corresponding signaling containing information about a real time feedback control of the dry corn milling process, based upon the signaling received.

2. Apparatus according to claim 1, wherein the signal processor or processing module is configured to provide the corresponding signaling as control signaling to provide the real time feedback control of the dry corn milling process.

3. Apparatus according to claim 1, wherein the one or more constituents of the output stream are selected from the following group:
corn oil,
corn syrup,
one or more proteins, and
leftover yeast solids.

4. Apparatus according to claim 1, wherein the measurement of the one or more constituents of the output stream are selected from the following group:
the purity of corn oil in the output stream, including the fat content;
the purity of the syrup in the output stream, including the carbohydrate content;
the amount of protein in the output stream, including proteins useful in animal foods;
the amount of water in the output stream; and
the amount of leftover yeast solids in the output stream.

5. Apparatus according to claim 1, wherein the measurement of the one or more constituents of the output stream is based upon an optical interrogation of the output stream.

6. Apparatus according to claim 5, wherein the optical interrogation of the output stream includes processing optical signaling provided and sensed in relation to the output stream.

7. Apparatus according to claim 5, wherein the optical interrogation of the output stream includes using a near-infrared spectroscopy technique for optically interrogating the output stream.

8. Apparatus according to claim 5, wherein the optical interrogation of the output stream includes using a Raman optical scattering technique for optically interrogating the output stream.

9. Apparatus according to claim 1, wherein the apparatus comprises an optical measurement/interrogation device configured to provide optical interrogation signaling, receive sensed optical interrogation signaling containing information about the one or more constituents of the output stream and provide the signaling, including where the optical measurement/interrogation device includes an optical probe.

10. Apparatus according to claim 1, wherein the measurement of the one or more constituents of the output stream is based upon a chemical interrogation of the output stream.

11. Apparatus according to claim 10, wherein the chemical interrogation of the output stream includes processing a sample/portion of the output stream based upon determining a chemical content of the portion of the output stream.

12. Apparatus according to claim 11, wherein the apparatus comprises a chemical measurement/interrogation device configured to receive the sample/portion of the output stream containing the one or more constituents of the output stream, process the sample/portion and provide the signaling containing the chemical interrogation information about the one or more constituents of the output stream.

13. Apparatus according to claim 1, wherein the real time feedback control of the dry corn milling process includes providing a control signal to adjust one or more parameters of the dry corn milling process, including where the control signal is used to control the centrifuge, a backend process such as a fermentation process, or both.

14. Apparatus according to claim 13, wherein the one or more parameters include some combination of the following:
a chemical parameter adjustment to one or more sub-processes in the dry corn milling process, including an adjustment to the amount, or timing, or location, of a de-emulsifier dosed to the centrifuge; or
a lever parameter adjustment of one or more components in the dry corn milling process, including adjusting a control or throughput lever in the centrifuge; or
a throughput parameter adjustment of one or more components in the dry corn milling process, including adjusting a throughput in the centrifuge; or
a flow parameter adjustment in the one or more components in the dry corn milling process, including adjusting a flow parameter of the centrifuge; or
a cycle time parameter adjustment in the one or more components in the dry corn milling process, including varying the cycle time of the centrifuge; or
a backflush parameter adjustment in the one or more components in the dry corn milling process, including setting up backflush cycles for the centrifuge; or
a diversion parameter adjustment in the one or more components in the dry corn milling process, including diverting a portion of the output stream of the centrifuge to another component in the dry corn milling process.

15. Apparatus according to claim 1, wherein the centrifuge is configured to receive an input stream containing syrup/oil and provide a co-product stream containing the one or more constituents having corn oil and light, low density solids.

16. Apparatus according to claim 15, wherein the centrifuge is configured to receive the input stream and provide a second co-product stream containing the one or more constituents having corn syrup and higher density solids.

17. Apparatus according to claim 8, wherein the Raman optical scattering technique includes comparing a sensed optical scattering signaling in the output stream to a signature optical scattering signaling stored in an optical scattering database and determining the purity of corn oil in the output stream based upon the comparison.

18. Apparatus according to claim 13, wherein the real time feedback control of the dry corn milling process includes sensing a desired level of corn oil capture, and providing the control signal to divert a portion of corn oil from the output stream to a distillation process in the dry corn milling process for producing dried distillers grain (DDGs) that goes into animal feed to increase its fat content.

19. Apparatus according to claim 13, wherein the real time feedback control of the dry corn milling process includes controlling a split of corn oil and corn syrup provided from the centrifuge, and determining whether to feed either the corn oil or the corn syrup back to the centrifuge for further purification, or claim the corn oil, or sending some portion of the corn oil to a backend process of the dry corn milling process.

20. A method comprising:
receiving in a signal processor or processing module signaling containing information about a measurement of one or more constituents of an output stream from a centrifuge in a dry corn milling process, including to produce Ethanol; and
determining in the signal processor or processing module corresponding signaling containing information about a real time feedback control of the dry corn milling process, based upon the signaling received.

21. A method according to claim 20, wherein the method also comprises providing from the signal processor or processing module the corresponding signaling as control signaling to provide the real time feedback control of the dry corn milling process.

22. A method according to claim 20, wherein the one or more constituents of the output stream are selected from the following group:
corn oil,
corn syrup,
one or more proteins, and
leftover yeast solids.

23. A method according to claim 20, wherein the measurement of the one or more constituents of the output stream are selected from the following group:
the purity of corn oil in the output stream, including the fat content;
the purity of the syrup in the output stream, including the carbohydrate content;
the amount of protein in the output stream, including proteins useful in animal foods;
the amount of water in the output stream; and
the amount of leftover yeast solids in the output stream.

24. A method according to claim 20, wherein the measurement of the one or more constituents of the output stream is based upon an optical interrogation of the output stream.

25. Apparatus comprising:
means for receiving in a signal processor or processing module signaling containing information about a measurement of one or more constituents of an output stream from a centrifuge in a dry corn milling process, including to produce Ethanol; and
means for determining in the signal processor or processing module corresponding signaling containing information about a real time feedback control of the dry corn milling process, based upon the signaling received.

26. Apparatus according to claim 25, wherein the apparatus also comprises means for providing the corresponding signaling as control signaling to provide the real time feedback control of the dry corn milling process.
